(19) United States
(12) Patent Application Publication
(10) Pub. No.: US 2025/0205153 A1
(43) Pub. Date: Jun. 26, 2025

(54) INHALATION COMPOSITE AND CARRIER BASED FORMULATION COMBINATION

(71) Applicant: HOVIONE SCIENTIA LIMITED, Cork (IE)

(72) Inventors: Susana SALDANHA, Lisboa (PT); Luis SOUSA, LISBOA (PT); Slavomira DOKTOROVOVA, LISBOA (PT)

(21) Appl. No.: 18/852,256

(22) PCT Filed: Mar. 30, 2023
(86) PCT No.: PCT/GB2023/050841

§ 371 Date: Sep. 27, 2024

(30) Foreign Application Priority Data
Mar. 31, 2022 (PT) 117893

(51) Int. Cl. A61K 9/00; A61K 9/16; A61K 31/496; A61K 31/675; A61M 15/00

(52) U.S. Cl. CPC A61K 9/0075; A61K 9/1617; A61K 9/1623; A61K 9/1694; A61K 31/496; A61K 31/675; A61M 15/0028; A61M 2202/064

(57) ABSTRACT

An inhalable pharmaceutical composition comprises: (i) Spray dried cohesive composite active particles, each composite active particle comprising an active pharmaceutical ingredient (API) material and an excipient; and, (ii) carrier particles Wherein the composite active particles and the carrier particles are blended in a physical mixture. A process of preparing the pharmaceutical composition comprises the steps of: i) providing a solution of an active pharmaceutical ingredient (API) material and a solution of an excipient, which may be separate solutions or a combined solution, and spray drying to provide cohesive composite active particles, wherein each composite active particle comprises an active pharmaceutical ingredient (API) material and an excipient: ii) blending the spray dried cohesive composite active particles with carrier particles to form a physical mixture. The composition may be used in a dry powder inhaler (DPI), particularly a reservoir DPI device.

The present invention relates to an inhalable pharmaceutical formulation that enhances the dosing efficiency either by increasing the active ingredient solubility and/or by increasing the aerodynamic performance of the spray dried particles. More particularly, it relates to methods of producing said formulation by a combination of spray drying and blending unit operations. Moreover, the present invention provides a solution for the processability of highly cohesive powders such as spray drying powders. The pharmaceutical composition can be applied in the pharmaceutical field more specifically in high drug load inhalable powders or for insoluble active ingredients.

### BACKGROUND OF THE INVENTION

The present invention relates to an inhalable pharmaceutical formulation that enhances the dosing efficiency either by increasing the active ingredient solubility and/or by increasing the aerodynamic performance of the spray dried particles. More particularly, it relates to the methods of production of said formulation by a combination of spray drying and blending unit operations. Moreover, the present invention provides a solution for the processability of highly cohesive powders such as spray drying powders. The pharmaceutical composition can be applied in the pharmaceutical field more specifically in high drug load inhalable powders or for insoluble active ingredients.

Carrier based formulations and composite particles for inhalation are both widespread solutions for delivery of active ingredients to the lungs in the form of powder. Powder-based inhalers have been used mostly for treatment of chronical respiratory diseases such as asthma or chronic obstructive pulmonary disease. Nevertheless, in the past recent years dry powder formulations have deserved considerable attention for acute respiratory treatments such as infectious diseases or vaccines. This led to an increase of interest in delivering to patients high payloads of pharmaceutical compounds such as antibiotics, antivirals, vaccines, proteins, peptides and other drugs that can act systemically, administered through the lung. Such compositions with a high drug load are typically characterized by highly adhesive and cohesive powders resulting from low median particle size, in addition to low bulk density, typically in the range of 0.1 g/cm³ to 0.5 g/cm³, especially for the composite engineered particles.

Therefore, considering the powders characteristics, we have appreciated it is a major challenge to fill these powders into devices for inhalation delivery. Frequently they are associated with long filling process times, high variability between doses and increased challenges in choosing the filling principle. These challenges lead to time-consuming operations at an industrial scale and lack of efficiency and reproducibility.

Even more, we have appreciated it is also challenging to aerosolize the powder efficiently, minimizing the retention into the device, especially in medium to high resistance devices, such as reservoir DPI devices.

The following discussion of the prior art is intended to present the invention in an appropriate technical context and allow its significance to be properly appreciated.

Pulmonary drug delivery through Dry Powder Inhalers (DPIs), present several advantages by virtue of its propellant free nature, high patient compliance, high dose carrying capacity and drug stability. This led to a rapid development in the recent past to realize the full potential of lungs for local and systemic treatment of diseases. Nevertheless, DPIs are complex in nature and their performance relies on many aspects including the design of inhaler, the powder formulation and the airflow generated by the patient.

The aerosol particles inhaled from a DPI would exhibit different sizes, leading to differences in regional lung deposition, resulting in variations in therapeutic effect. The fine-particle component of aerosols is the fraction that leads to a therapeutic effect, and it is defined as the percentage of particles that are smaller than 5 μm aerodynamic diameter, or, in the case of certain particle-sizing instruments, a cut-off diameter that is close to 5 μm. Thus, degree of dispersibility is an important consideration for both the quality and efficacy of pharmaceutical aerosols. The fine particle dose (FPD) is the mass, in milligrams, of particles with a cut-off diameter smaller than 5 μm. The fine particle fraction (FPF) is the fine particle dose divided by the total emitted dose.

The highly cohesive nature of micronized active materials prevents accurate metering of the low doses required for inhaled drug products and renders the material difficult to handle during the manufacturing process. To overcome the issues of handling and dosing and to optimize the fluidization properties of DPI formulations, a micronized API is typically blended with a coarse material that can be easily aerated and fluidized. For carrier-based DPI formulations, the material is usually a coarse-sized fraction of a sugar, such as lactose monohydrate. There are several examples in the carrier-based field, where the micronized active ingredient is blended with sugars, namely lactose.

### Carrier Based Formulations

US 2017/0266122 A1 describes a pharmaceutical composition comprising composite active particles of active material and magnesium stearate, and carrier particles for pulmonary delivery. EP 1913939 A1 describes a method for making particles composed of active ingredient and excipient, comprising a co milling step and blending of the co milled particles with a carrier. EP 1617820 B1 describes a dry powder inhaler comprising a formulation having a co milled active ingredient with an excipient (jet milled composite particles) which are additionally attached to a carrier. These are common carrier-based formulations, having the milled active ingredient combined with the excipient by dry coating and/or dry milling. This excipient is used to reduce the cohesion between the milled particles. These disclosures differ significantly from the present invention since the composite particle described therein have substantially distinct characteristics given the considerable differences on their manufacturing method when compared to the present invention. The manufacturing process disclosed in these publications for producing composite particles is jet milling, meaning dry milling. By contrast, the manufacturing process proposed in the present invention is spray drying, and does not involve a milling step, which brings several advantages in terms of particle engineering. Even more, the milled particles in publications US 2017/0266122 A1, EP 1913939 A1 and EP 1617820 B1 are crystalline particles which contrasts with the amorphous spray dried particles proposed in the present invention. The spray dried particles proposed in the present invention have optimized and customized physico-chemical characteristics that present several advantages when compared to milled active material.

WO 2005/025536 describes a method of making composite active particles by jet milling active particles in the presence of an additive material. This patent application's scope regards a different manufacturing process for the composite active particles when compared with the present invention. This publication uses a common milling step in contrast with the proposed invention method of manufacturing (spray drying), this significant difference between methods leads to significantly different final composite particles, as stated before.

EP 2821061 A1 patent describes a dry powder formulation comprising a micronized active agent and a carrier agent, wherein fine particles and coarse particles of carrier agent are employed. This approach is well known in the pharmaceutical industry for dry powder inhalers since it enables a significant increase on fine particle delivery of the active materials.

Nevertheless, this publication differs significantly from the present invention considering that those improvements are suitable for low dosage milled particles, examples show 0.05 to 4.5% w/w of active ingredient. It is common knowledge in the field of respiratory formulations that, for carrier-based formulations, increasing the concentration of active ingredient in the blend results in increasing numbers and size of individual agglomerates and densely packed active ingredient multi-layers on the surface of the lactose carrier. The active ingredient present within the multi-layer does not disperse as individual primary particles but as dense agglomerates, which leads to a decrease in aerosol performance and decreased fraction deposited in lung (fine particle fraction).

We have appreciated that carrier based formulations thus present drawbacks especially for high drug loads (>10% w/w active material). Several studies reveal that as the active material concentration in the blends increases, aerosol performance of the formulation decreases, in an inversely proportional manner.

### Spray Dried Composite Particles

The vast majority of commercialized DPIs depend on the use of a physical mixture of a coarse carrier (most commonly lactose monohydrate sized 50-100 μm) with small inhalable drug particles (1-5 μm) in order to overcome the strong cohesive and adhesive properties of the drug and to improve metering. This balance must be optimized in order to successfully deliver the dose to the deep lung. Unfortunately, this balance is difficult to achieve due to the complexity of the interparticle forces which depends on several intrinsic and extrinsic factors.

Thus, it has become commonly acceptable to achieve a deep lung deposition of only 30% of the active ingredient dose. Such a low percentage, in addition to the high mass of coarse carrier used, is a serious hindrance to the use of DPIs for delivering high-dose drugs.

In the last decade, performance of DPIs has improved significantly using engineered drug particles by spray drying (from now on spray dried particles), carrier free and with modified excipient systems. Spray dried particles can comprise the active ingredient only or can comprise the active ingredient plus at least one excipient (spray dried composite particles).

Spray drying started to be explored in the 1980s in inhalation as an alternative method of making fine particles with desirable flow and dispersion characteristics, without the need of using coarse carriers.

Spray drying is a tool to transform the physiochemical features of a material in order to improve and optimize its aerosol performance as well as dissolution characteristics. The solid starting material is dissolved in a liquid medium in order to be reconstructed again with well-controlled characteristics at the particle level (size, morphology, polymorphic form, density and composition) and at the powder level (flowability, dispersibility and aerosolization). Several excipients have been tested in spray dried inhalation powders for different purposes such as bulking, surface modification, and pore forming agents which have a crucial role to play both in optimizing aerodynamic performance and stabilizing the particles' physicochemical properties (spray dried composite particles).

Composite particles produced by spray drying are disclosed for example inUS 7862834 B2 which describes a spray dried pharmaceutical formulation comprising particles of active ingredient and at least one excipient as well as the method of manufacturing said particles.

The particles described therein are composite particles comprising the active ingredient and an excipient which at least partially encapsulates said active agent. The particle mentioned significantly differs from the present invention considering that the present method of production typically leads to an amorphous spray dried particle which means that the active material is molecularly dispersed in at least one excipient, both being in the amorphous state. Even more, the scope of the present invention is broader than U.S. Pat. No. 7,862,834 B2 in that it aims to solve the processability and industrialization problems related to the low density and high cohesiveness of spray dried composite particles. Due to the overall small particle size of spray dried formulations for respiratory administration, the handling and processing of these powders is challenging. The large surface area leads to uncontrolled agglomeration and may hinder the filling process. Furthermore, spray dried materials are oftentimes less dense and of low mechanical stability, thus, the filling process itself can have a negative impact on the success of inhalation, as the powder gets compacted and may not be well re-dispersed. The present invention addresses or substantially minimizes these problems.

Publications WO0062819 (A1), KR20010034594 A, CA2265198 (A1) and EP1925295 (A1) describe composite formulations of active ingredient (methacholine, histamine, nicotine or others) and a sugar prepared by dissolving both components in a suitable solvent or dispersing to create a uniform solution and drying the solution by spray drying to form a powder. No additional carrier component is disclosed, and this would in fact be contrary to their teaching.

In publication WO2021234366 A1, the compositions are provided in the form of amorphous, mono-particulate powders. The particles of the powdered compositions described are thus presented as an amorphous composite of active ingredient, the carrier materials and, optionally, other ingredients. They are not composed of physical associations of two or more discrete, separate sets of particles of different ingredients in the form of a mixture, such as an ordered, or interactive, mixture of smaller particles of active ingredients associated with larger, but separate and chemically distinct, particles of carrier substances. The manufacturing process mentioned on this patent refers to dissolving the API and the excipients in a solvent and spray dry the solution, which leads to a molecularly mixture of API and excipients.

The development of high dose DPIs continues to be hampered by the strong and variable inter-particulate forces that occur between the tiny spray dried composite particles. Such forces hinder powder flow during processing and dosing and lead to the formation of large intractable aggregates that can prevent the active ingredient reaching the lower airways causing variable or low fine particle fractions.

The powder dispersion process in a DPI is highly complex and involves several physical mechanisms, namely air turbulence and shear flow-induced aerodynamic forces, particle-device impactions within the inhaler body, mechanical vibration and particle-particle collisions.

We have also appreciated that another point for improvement is the aerodynamic performance of DPIs when handling devices which rely only on the turbulence generated by the airflow to aerosolize the powder, such as reservoir and blister-based devices. Thus far, there is no clear or optimized formulation available for improving the aerodynamic performance in such devices.

None of the aforementioned publications solve all the challenges that the present invention proposes to solve, related to inhalable products. These challenges include: to increase the fine particle fraction, especially in reservoir and/or blister-based devices, to increase the solubility (in case of poorly soluble drugs), and to overcome the difficulties related to processability and industrialization.

### DESCRIPTION OF THE INVENTION

The present invention seeks to provide an inhalable pharmaceutical formulation that comprises spray dried cohesive composite particles physically blended with carrier particles. The present invention proposes to enhance the dosing efficiency by increasing the active ingredient solubility and/or by increasing the aerodynamic performance of the spray dried particles. The increasing solubility is achieved through the amorphous state of spray dried composite particles. On the other hand, the aerodynamic performance increase is obtained by physically blending the spray dried composite particles with a carrier. Furthermore, the present invention seeks to provide a method of production said formulation by combination of spray drying and blending unit operations. Moreover, the present invention proposes a solution for processability of highly cohesive powders such as spray drying powders. Furthermore, the present invention presents a solution for the poor aerosolization of spray dried composite particles, especially in high resistance devices. The pharmaceutical composition can be applied in the pharmaceutical field more specifically in high drug load inhalable powders or in insoluble active ingredients for respiratory intake.

In contrast to the disclosures of WO0062819 (A1), KR20010034594 A, CA2265198 (A1) and EP1925295 (A1), in the present invention the composite particles (composed of the active ingredient and at least one excipient) produced by spray drying are physically blended with a carrier, by low or high shear mixing after the spray drying unit operation. In consequence, the final powder characteristics are substantially different. In the present invention, the composite particles typically have a rather small particle size (Dv90 below 10 μm) and the carrier particles typically have a higher particle size (Dv90>10 μm), as opposed to the particles described in publications WO0062819 (A1), KR20010034594 A, CA2265198 (A1) and EP1925295 (A1) where the composite particles of both active ingredient (methacholine, histamine and nicotine) and sugar (carrier) have a Dv90 below 10 μm. Furthermore, the formulation of the present invention has the advantage of being a blend with an adequate cohesive-adhesive balance between the composite particles and the carrier particles, such that the composite particles (comprising the active ingredient) are release from the carrier upon actuation. The carrier thus stays in the throat and the composite particles flow to the airways. On the contrary, the formulations described in WO0062819 (A1), KR20010034594 A, CA2265198 (A1) and EP1925295 (A1), since these are composite particles only, the active ingredient (methacholine, histamine, nicotine or others) will be delivered to the alveoli and the lower airways of a patient together with the carrier.

In contrast to WO2021234366, the present formulation physically blends at least two different types of particles, the spray dried composite active particles, preferably with a small particle size suitable for inhalation (for example, a dv90≤10 μm) and a carrier, preferably with a larger particle size, for example a dv90≥10 μm. Notably, in this field, it is challenging to find a suitable solvent to dissolve both the active ingredient and the excipients, especially if the former is poorly soluble. In the process of the present invention, the composite particles are manufactured prior to the blending with the carrier material, such as a sugar, which means that there is greater freedom to choose the solvent. This is a further advantage of the present invention.

According to one aspect of the present invention, there is provided an inhalable pharmaceutical composition comprising:
(i) Spray dried cohesive composite active particles, each composite active particle comprising an active pharmaceutical ingredient (API) material and an excipient; and,
(ii) carrier particles
wherein the composite active particles and the carrier particles are blended in a physical mixture.

Other aspects of the invention are described below.

The terms cohesive/cohesion are well known in the art of pharmaceutical powders, and are commonly associated with a powder's flowability, which is often the most influential property in regard to bulk powder behaviour. Cohesion is a mechanism that acts between particles and has the tendency to ‘bond’ one particle to its neighbor.

The terms “spray dried cohesive composite active particles” and “composite active particles” are used interchangeably herein.

The carrier particles are separate and distinct from the composite active particles. There are thus two populations of particles in the present invention. These are mixed or blended into a physical mixture once each particle population has been obtained. Thus, they are not co-produced.

An inhalable composition is one that is suitable for administration via the inhalation route i.e. capable of being inhaled via the nasal or buccal passages of a patient in need thereof. Preferably, a dry powder inhaler, such as a reservoir device, is employed to administer the composition to the airways.

### BRIEF DESCRIPTION OF THE DRAWINGS

FIG. 1 illustrates the spray-drying set up used;

FIG. 2 illustrates a SEM image of spray dried composite particles from Example 1;

FIG. 3 illustrates a XRPD diffractogram of the spray dried composite particles from Example 1;

FIG. 4 illustrates a DSC diffractogram of spray dried composite particles from Example 1;

FIG. 5 illustrates the dissolution curves of spray dried composite particles and jet milled micronized material from Example 1.

FIG. 6 illustrates the X-ray powder diffraction pattern of the spray dried composite particles obtained in Example 2.

The composite active particles used in the inhalable pharmaceutical composition of the invention preferably have a mass median aerodynamic diameter (MMAD) of equal to or less than less than 10 μm, more preferably less than 5 μm. The MMAD may for example range from 1 to 10 μm, or from 1 to 8 μm, or from 2 to 6 μm, or from 1 to 5 μm.

In one aspect, solubility and dissolution rate of the active pharmaceutical ingredient (API) material is higher than that of the crystalline isolated form of the API. The solubility and dissolution rate is typically measured in an aqueous media or solution. For example, the solubility and dissolution rate may be measured in HBSS (Hank's Balanced Salt Solution).

This is useful as it can be employed to mimic lung fluid.

In a preferred aspect, the composite active particles comprise active pharmaceutical ingredient (API) material in an amorphous form.

In one aspect, the composite active particles, particularly when the API is in amorphous form, are more soluble than particles of active pharmaceutical ingredient (API) material alone.

The composite active particles may further comprise active pharmaceutical ingredient (API) material in a crystalline form. Thus, there may be a mixture of both amorphous API and crystalline API. Typically, at least 50% or more, or at least 70% or more, or at least 90% or more, or at least 95% or more of the total API will be in amorphous form, In one aspect, 99% or more, or 100% of the API is in amorphous form.

The excipient present in the composite particles may in amorphous form or may be in crystalline form. One or more excipient component may be in crystalline form, and one or more excipient component may be in amorphous form. For example, the amino acid component, such as leucine, may be in crystalline form. Whereas the sugar component, such as trehalose, may be in amorphous form. Typically, where an amorphous form is employed, at least 50% or more, or at least 70% or more, or at least 90% or more, or at least 95% or more of that excipient will be in amorphous form. In one aspect, 99% or more, or 100% of that excipient is in amorphous form. The spray drying process can be used to obtain both API and excipient in amorphous, or substantially amorphous, form. One preferred aspect includes leucine in crystalline form and trehalose in amorphous form.

In one aspect of the present disclosure, the composite active particles comprise about 10 to about 90% of active pharmaceutical ingredient (API) material by weight of the composite active particles. More preferably the composite active particle comprise 50 to 80% of API.

In a further aspect, in the pharmaceutical composition of the invention, the excipient comprises an amino acid, or a sugar, or a mixture of an amino acid and a sugar. One or more amino acids may be used. One or more sugars may be used.

The amino acid component may for example comprise leucine, tryptophan, alanine, valine, isoleucine, trileucine, dileucine, methionine, phenylalanine, or proline or a mixture of two or more thereof. Preferably, the amino acid component comprises leucine, isoleucine, trileucine or dileucine or a mixture of two or more thereof. Leucine is often preferred. An enantiomer may be used, for example L-Leucine, or D-Leucine.

The sugar component may comprise any suitable sugar. Examples are the monosaccharide and disaccharide sugars. In some aspects, preferably the sugar comprises a disaccharide.

For example, the sugar may comprise trehalose, lactose, mannitol, or sucrose or a mixture of two or more thereof.

In one preferred aspect, the sugar preferably comprises trehalose, lactose, or sucrose or a mixture of two or more thereof. Trehalose is often preferred.

A combination of a form of leucine (which may for example be leucine, isoleucine, trileucine or dileucine, L-Leucine, or D-Leucine) and trehalose is one preferred excipient. Trehalose or a derivative thereof may also be used alone as the excipient.

In a further aspect of the present disclosure, the spray dried cohesive composite active particles comprise about 10 to about 85% of the pharmaceutical composition by weight. In a more preferred embodiment, the spray dried composite active particles comprise about 50 to about 85% of the pharmaceutical composition by weight.

The carrier particles, which are a discrete population of particles, and separate from the spray dried cohesive composite active particles, may for example be selected from a group comprising lactose, mannitol, trehalose, raffinose, sucrose, microcrystalline cellulose or a mixtures of two or more thereof. In one preferred aspect, the carrier particles preferably comprise lactose and more preferably lactose monohydrate, or a mixture of lactose and lactose monohydrate.

In one aspect, the carrier particles comprises about 15% to about 90% of the pharmaceutical composition by weight. In a more preferred embodiment, the carrier particles comprise about 15 to about 50% of the pharmaceutical composition by weight.

In a preferred aspect, the mass median aerodynamic diameter (MMAD) of the carrier particles is greater than that of the spray dried cohesive composite active particles. Preferably, the MMAD of the carrier particles is greater than 10 μm, more preferably greater than 25 μm, or greater than 50 μm. An MMAD ranging from about 50 μm to about 100 μm has been found to give good results. For example, a range of about 50 μm to about 75 μm may be used. We have found an advantage resulting from the MMAD of the carrier particles being significantly larger than the MMAD of the spray dried cohesive composite active particles.

In a further aspect of the present invention, there is provided a process of preparing a pharmaceutical composition as described herein, which process comprises the steps of:
i) providing a solution of an active pharmaceutical ingredient (API) material and a solution of an excipient, which may be separate solutions or a combined solution, and spray drying to provide cohesive composite active particles, wherein each composite active particle comprises an active pharmaceutical ingredient (API) material and an excipient;
ii) blending the spray dried cohesive composite active particles with carrier particles to form a physical mixture.

As described above, the spray dried cohesive composite active particles and the carrier particles are two separate, discreet populations of particles.

In the presently disclosed process, after step (i) or after step (ii), or both, a post drying or conditioning step may be performed.

In a further aspect of the process, the composite active particle size distribution may be controlled to obtain the desired distribution. For example, the composite active particle size distribution may be controlled to a Dv50 value of equal to or <5 μm.

In an additional aspect therefore, the invention provides spray dried cohesive composite active particles having a Dv50 value of from about 1 to about 3 μm.

In step (ii) of the process, the blending may comprise high shear or a low shear blending process. Typically low shear blending is used and we have found this gives good results.

In a further aspect, the invention provides a blister or series of blisters, or one or more capsules, for use in a dry powder inhaler (DPI), comprising a pharmaceutical composition according to the invention described herein. As will be understood, this may be achieved by methods known in the art wherein the pharmaceutical composition is filled into blisters, or capsules; or devices such as reservoir type dry powder inhalers.

Accordingly, there is also provided a dry powder inhaler (DPI) comprising a blister or series of blisters, or one or more capsules, which comprise a pharmaceutical composition according to the invention as described herein.

The invention also provides a dry powder inhaler (DPI) which relies on turbulence from air flow to mobilise the dry powder, for example a reservoir device, comprising a pharmaceutical composition according to the invention as described herein. As will be understood, several inhaler devices, the primary example being a DPI reservoir device, rely on turbulence from air flow to mobilise the dry powder. The pharmaceutical composition described herein may thus be used with particularly good results with such devices, such as medium to high resistance drug DPI device, for example a DPI reservoir device, alternatively known just as a reservoir device; or a capsule-based device.

The present invention also provides a pharmaceutical composition according to the invention described herein for use as a medicament. As will be understood, the API component of the composition may be employed to treat a range of medical conditions. Administration to a patient is preferably via a dry powder inhaler, as described herein.

The invention is of particular utility in treating respiratory conditions or disorders. Accordingly, the invention also provides a pharmaceutical composition according to the invention described herein for use in the treatment of a pulmonary condition.

According to the present invention, an inhalable pharmaceutical composition is provided, comprising spray dried cohesive active particles and carrier particles, both of them physically blended. Furthermore, the solubility of active material of said composite particles is higher than its crystalline micronized form. This formulation strategy presents several advantages in the inhalation field. On one hand, the formulation presents an increased dosing efficiency through increment of solubility and improved aerosolization when compared to the crystalline micronized material. The aerosolization, meaning the fine particle dose, is higher even compared with the spray dried composite particles alone, using high resistance devices, such as DPI reservoir devices. On the other hand, the blend presents physical properties, such as flowability and cohesiveness that promotes a smoother capsule filling manufacturing process when compared with the composite particles alone. Said properties lead to a leaner scale up and industrialization. The present invention can be applied for low solubility compounds, promoting a higher bioavailability as a consequence of the higher dosing efficiency. Furthermore, the present invention can be applied for labile compounds that do not stand up well to milling techniques.

The spray dried composite particles provided by the present invention differ significantly from the prior art in at least two major features. On one hand, they can be amorphous (both active ingredient and at least one excipient), which leads to an increased dissolution rate of the active ingredient, and consequently lower dose and higher therapeutic effect. This allows the administration of higher drug loads to the lungs which is mandatory in case of pharmaceutical compounds for acute treatments, such as, but not limited to, antibiotics, antivirals, vaccines, proteins and peptides. On the other hand, the spray dried composite particles of the present invention contain the excipient as an intrinsic part of the primary particle, which is ideal to prepare inhalable formulations with improved and reproducible physical and chemical characteristics. Even more, said composite particles are especially useful for particle engineering of labile active ingredients that cannot be milled through the traditional technique due to their physical and/or chemical degradation. It is current knowledge that the composite particles in the prior art are associated with increased drug delivery by themselves, yet this is not observed in reservoir and blister-based devices, where the dispersion mechanism is not efficient or strong enough to disaggregate such cohesive spray dried composite particles. In that case, the present invention presents a significant advantage when compared with the composite particles alone. The formulation provided by the present invention proves to have a better aerodynamic performance when compared with the spray dried composite particles alone.

The present invention is not a common carrier-based formulation, where the crystalline API is blended with fine and coarse lactose. The object of the present invention is a combination of two independent formulation techniques, which the prior art indicates were not supposed to be combined. Composite particles described in the prior art arose from the need to solve the issues that the carrier-based formulations could not solve. It is not a straightforward approach to combine such techniques, and there is no prior disclosure of this being done. Furthermore, all prior described composite formulations are claimed to be carrier free, which in the prior art is stated as the biggest advantage of the composite particles. In contrast to this, the present invention has shown that this is not true for all formulation and device combinations, and that there are cases where a combination of spray dried composite particles plus carrier particles is beneficial.

The process of preparing the present invention's spray dried composite particles is well known and described in the literature. The preparation of the spray dried particles involves the dissolution of the active ingredient and excipients in suitable solvent or mixture of solvents. Any appropriate concentration of active ingredient and excipient or mixture of excipients may be used, up to the solubility limit. The particles of active ingredient and excipient are obtained by solvent evaporation through spray drying or other suitable technique such as freeze drying, to be performed using any suitable or commercially available equipment. A variety of atomization methods can be used, depending on the equipment chosen, for example two- or three-fluid nozzle, pressure or ultrasonic nozzles. The preferential atomization gas flow in terms of liters per hour can be adjusted to the equipment in use and any suitable atomization gas flow can be used. Typically, for a small scale unit, 150 to 300 milliliters per hour is preferred. On an industrial scale a different flow may be used. Any suitable drying temperature can be used, ranging from about 30° C. to about 220° C. The inlet temperature may be adjusted to attain the desired outlet temperature. Any suitable solution flow rate can be used. The outlet temperature, atomization flow rate, solution concentration and solution flow rate, among other parameters, can be combined and adjusted to obtain a compound with suitable quality. The spray dried composite active particles obtained are amorphous and stable throughout time. The particle formation process in the spray dryer is controlled in order to obtain the desired particle size. The range usually defined for this type of drying technique is below 25 μm and above 1 μm of medium particle size distribution, leading to a powder with low density, usually between 0.1 g/m³ and 0.5 g/m³, and high cohesiveness.

In a preferred embodiment the spray dried composite active particles have a particle size suitable for inhalation, meaning a mass median aerodynamic diameter of less than 10 μm, more preferably less than 5 μm. In another embodiment, the spray dried composite active particles are amorphous, and its dissolution rate is higher than the active ingredient crystalline isolated form. In a preferred embodiment the composite active particle comprises an active pharmaceutical ingredient (API) from the following therapeutic groups, but not limited to, antibiotics, antifungal agents, antiviral agents, antipsycotic agents, immunosuppressants, bronchodilators, anti-parkinsonian agents, anti-inflamatory or anti-cancer drugs. Examples of active ingredients useful in this invention include but are not limited to Streptomycin, Isoniazid, para-aminosalicylic acid, tobramycin, gentamycin, rifampicin, pyrazinamide, ethambutol, colistin, aztreonam, ciprofloxacin, amoxicillin, fluoroquinolone, cefuroxime, cefpodoxime, itraconazole, voriconazole, pentamidine, bevacizumab, paclitaxel, ceritinib, tacrolimus, fluticasone, salmeterol, salbutamol, beclomethasone, levodopa, loxapine, remdesivir, amantadine, ribavirin, zanamivir, rimantadine, oseltamivir, acyclovir, foscarnet, peramivir, baloxavir marboxil, ipratropium bromide, aclidinium bromide, tiotropium bromide, revefenacin, pirfenidone or nintedanib.

In a preferred aspect, the API is an antibiotic or is an antiviral compound. For example, the API may be a broad-spectrum antiviral compound, such as remdesivir. The antiviral compound may be a protide compound (a prodrug of a nucleotide), which is able to diffuse into cells. In another preferred aspect, the API may be an antibiotic, such as an ansamycin antibiotic. Ansamycins are a family of bacterial secondary metabolites that show antimicrobial activity against many Gram-positive and some Gram-negative bacteria, and include various compounds, including streptovaricins and rifamycins. The Ansamycin antibiotic compound may have an aromatic moiety, which can be a naphthalene ring or a naphthoquinone ring as in rifamycin and the naphthomycins, or another variation consists of benzene or a benzoquinone ring system as in geldanamycin or ansamitocin. Rifampicin is one preferred compound.

In one aspect, the API may exclude methacholine, or histamine, or nicotine, or a salt thereof. In one aspect, the composition of the invention may exclude anticholinergic compounds as the API. In particular, the composition may exclude a compound as shown below as the API, where X⁻ denotes a negatively charged anion:

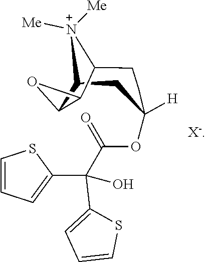

In one aspect, the composition of the invention may exclude an organic, physiologically acceptable, sterically demanding acid as part of the spray dried composite particles; and/or as part of the separate carrier particle component. Such an acid (which may be excluded) may be selected from among ascorbic acid, a fruit or culinary acid and a mono-, di- or trivalent carboxylic acid.

In a preferred embodiment the spray dried composite active particles comprise an aminoacid, a sugar, or a mixture thereof. In a more preferred embodiment, the aminoacid comprises leucine, tryptophan, alanine, valine, isoleucine, trileucine, dileucine, methionine, phenylalanine, proline or a mixture thereof. In a more preferred embodiment the sugar comprises trehalose, lactose, mannitol, sucrose or a mixture thereof.

The formulation of the present invention is comprised of a spray dried composite active particle, representing between 10 and 85% of the total formulation weight, and carrier particles, representing 15 to 90% by weight of the total formulation, both physically blended. In a preferred embodiment, the carrier particles are selected from a group comprising but not limited to lactose, mannitol, trehalose, raffinose, sucrose, or microcrystalline cellulose or mixtures thereof.

The formulation of the present invention is produced through mixing the spray dried composite particles with the carrier. This is typically a physical mixing step, and any suitable mixing technique may be used. In a more preferred embodiment, the mixing step can be performed by high shear mixing or low shear mixing. The carrier particles preferably comprise lactose, mannitol, trehalose, raffinose, sucrose, or microcrystalline cellulose or mixtures thereof.

The formulation of the present invention can be filled into devices or capsules. More preferably, the formulation of the present invention can be filled into a reservoir or blister-based device. The present invention formulation approach brings a new area of interest by combining two opposite formulation techniques for improvement of drug delivery, especially in high resistance drug devices.

In summary, the method of producing the formulation according to the present invention may typically comprise the three following steps:
a. Spray dried composite particles manufacturing;
b. Blending spray dried composite particles with carrier particles;
c. Optionally, filling of the final mixture into suitable containers (blister cavities, capsules or reservoirs).

Certain specific aspects and embodiments of the present invention will be explained in more detail with reference to the following examples. The Examples are set forth to aid in understanding the invention but are not intended to, and should not be considered to, limit its scope in any way. The experiments reported were carried out using a BUCHI model B-290 advanced spray dryer.

### Example 1

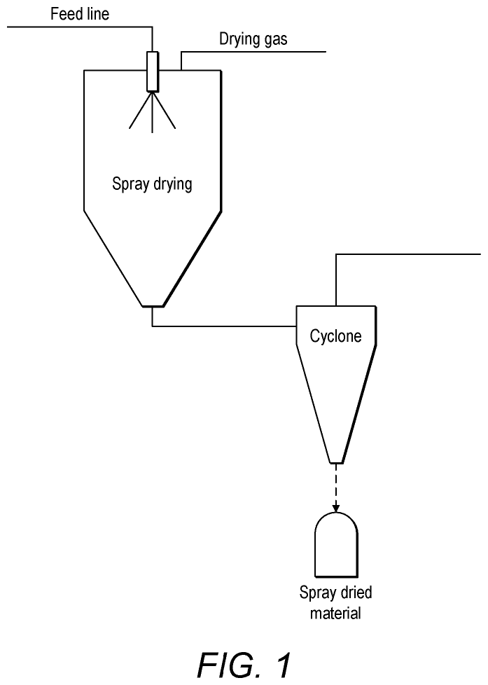

A formulation was prepared according to the present invention. For preparing the spray dried particles the active ingredient was dissolved and the excipients were dissolved in a suitable solvent. A lab scale spray dryer (Büchi, model B-290), equipped with a three fluid nozzle, was used to atomize and dry the solutions. Co-current nitrogen was used to promote the drying after atomization. The spray drying unit was operated in open cycle mode (i.e., without recirculation of the drying gas). FIG. 1 schematically shows the spray drying set up used. Before feeding the solution to the nozzle, the spray drying unit was stabilized with nitrogen to assure stable inlet (T_in) and outlet temperatures (T_out). After stabilization, the solution was fed to the nozzle by means of a peristaltic pump and atomized at the tip of the nozzle. The droplets were then dried in the spray drying chamber by current nitrogen. The stream containing the dried particles was directed into a cyclone and collected at the bottom. The main operating parameters during the spray drying process are summarized in table 1.

TABLE 1
Summary of the main operating conditions
Spray solutions

| | | |
|---|---|---|
| Remdesivir (REM) | 5.0 | g |
| Acetone | 398.0 | g |
| L-Leucine (LEU) | 2.5 | g |
| Trehalose (TRE) | 2.5 | g |
| Water | 597.0 | g |

Spray Drying parameters

| | | |
|---|---|---|
| T_in | 55 | ° C. |
| T_out | 40 | ° C. |
| F_drying (N2) | 40 | kg/h |
| Rotameter level | 50 | mm |

The compound obtained using the method of this invention is an amorphous solid with a higher dissolution rate compared with the corresponding crystalline form. Several tests confirm its amorphous form, such as x-ray powder diffraction (XRPD) or differential scanning calorimetry (DSC).

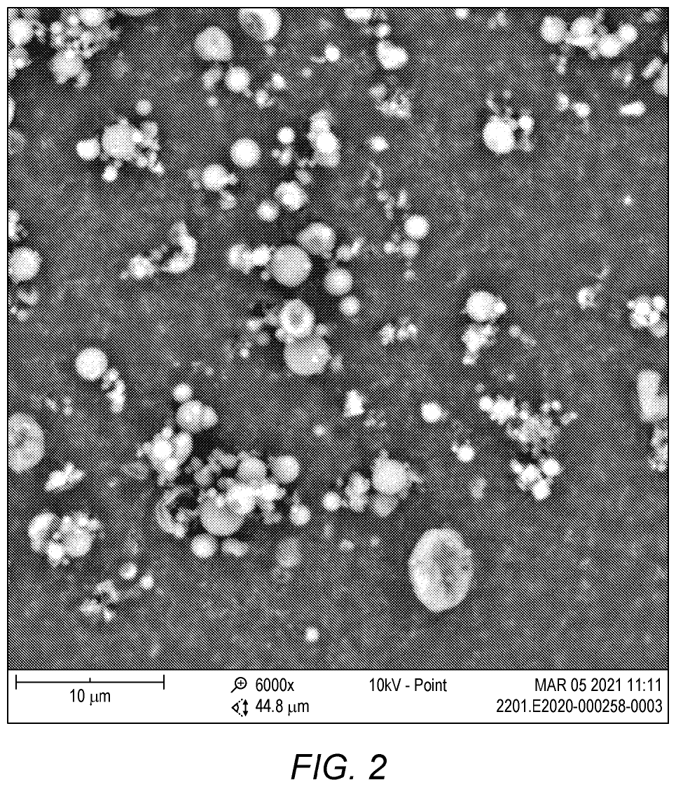

The appearance of the atomized material was characterized by means of scanning electron microscopy (SEM). A representative image of the particles obtained is shown in FIG. 2.

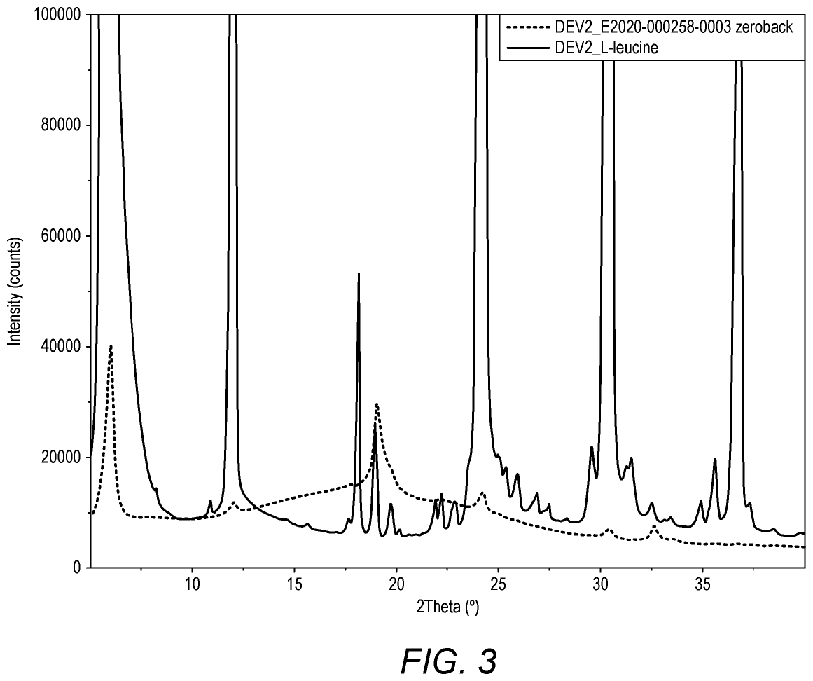

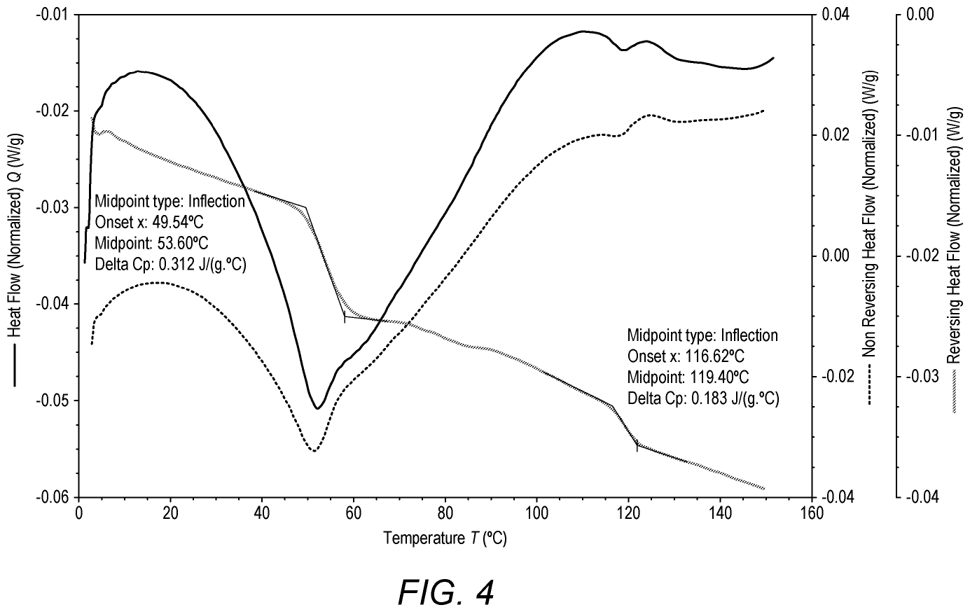

The X-ray powder diffraction pattern of the spray dried composite particles obtained according to the process herein described is presented in FIG. 3, this graph is overlaid with the L-Leucine crystalline particles in order to confirm that the peaks come from the L-Leucine, and the active ingredient is in the amorphous form. The Differential Scanning Calorimetry (DSC) from the spray dried composite particles obtained is presented on FIG. 4.

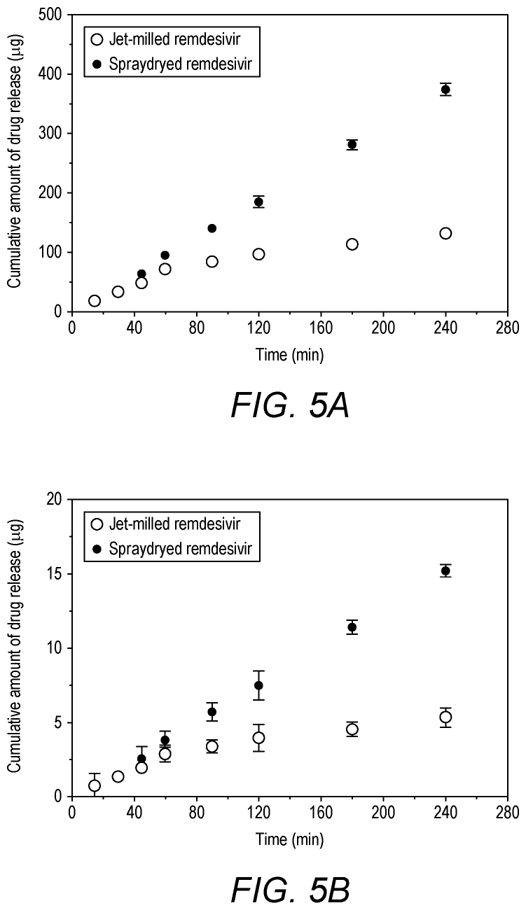

The composite active particles proved to have a higher dissolution, as measured by Franz cells apparatus, when compared to the crystalline micronized particles (obtained by jet milling), see for example FIG. 5. Dissolution profiles of the jet-milled and spray-dried remdesivir formulations were determined using the Franz cell diffusion apparatus described previously in the literature. Briefly, 2 mg of powder was weighed out on to filters and clamped on to each cell of the multi-station Franz cell (VB6, PermeGear Inc.). Each cell was contained within a heated water jacket that was preheated and then maintained at 37±0.05° C. via a circulating water bath, throughout the experiment. 23 mL of HBSS solution was placed in a water bath within each cell at the start of the experiment and pumped into the inlet port of the Franz cell at 5 mL/min via a multichannel peristaltic pump. The sampling port of the cell was connected to a second pump channel, which in turn returned the sample flow to the 23 mL sink. Constant agitation of the dissolution medium and renewal via the pumping ports ensured constant volume within the Franz cell and hence ensured the filter was uniformly wetted and sink conditions kept constant throughput the experiment. Samples of 0.6 mL were taken from the reservoir at predetermined time intervals (up to 24 h) and analysed in triplicate by HPLC.

The composite active particles obtained were blended with coarse lactose, the formulation composition is shown in Table 2. The spray dried composite particles and the coarse lactose were blended in a low shear blender, in two steps. The first step involved the blending of 66% w/w of total lactose with 50% w/w of the total amount of composite active particles, at 96 rpm, for 5 minutes. The second step involved the addition of the remaining lactose and composite active particles and blending at 96 rpm for 5 minutes more.

TABLE 2
Formulation composition

| Ingredient | Amount (g) | Amount (%) |
|---|---|---|
| Spray dried composite active particles | 5.0 | 50 |
| REM:LEU:TRE (50:25:25 ratio by weight) | | |
| Lactose monohydrate (coarse) | 5.0 | 50 |
| TOTAL | 10.0 | 100 |

The blend obtained was filled into a reservoir type device. The fill weight used was 31 mg. The filling was performed in an auger-filling principle equipment, and no clogging was observed. The filling time was less than 40 seconds per cavity, much lower than the spray dried composite particles alone. The aerodynamic performance of spray dried composite particles alone (A), composite blend (B) (i.e. the spray dried composite particles plus lactose) and carrier-based blend (C) (i.e. lactose plus micronized REM only) was assessed through Next Generation Impactor (NGI). The results are presented in Table 3. The emitted dose (ED) of the composite active particles alone was 2.5 mg as compared to the composite blend that was 7.9 mg. The majority of the powder stayed inside the device for the composite active particles alone (6.5 mg), whereas in the case of the composite blend, just a few micrograms of powder stayed in the device (0.8 mg). This shows that the flow through the reservoir device, for the composite particles alone, was not enough to fluidize the powder and make it get out. In vivo this might be even worse especially for conditions where the breath flowrate is compromised. The fine particle dose (FPD) of the composite blend is much higher than the composite active particles alone, 3.9 mg and 0.3 mg, respectively. This shows that not only is the emitted dose higher but also that the dose that reaches the lungs, and will have a therapeutic effect, is higher. This fine particle dose is higher even compared with a common carrier-based blend having the active ingredient micronized (i.e. the carrier-based blend, C).

TABLE 3
Aerodynamic performance by NGI

| | | | | C |
|---|---|---|---|---|
| | | A | B | Carrier- |
| | | Composite | Composite | based |
| | | particles | Blend | blend |
| Active material | | Composite REM | | Micronized |
| | | REM:LEU:TRE | | REM |
| | | (50:25:25) | | (25%) |
| Active material particle size | | | | |
| distribution(PSD) | | | | |
| Dv90 | | 4.3 | | 5.7 |
| Dv50 | | 1.7 | | 2.1 |
| Dv10 | | 0.6 | | 0.6 |
| Composition | | | | |
| Composition | Composite | 100% | 50% | 0 |
| | particles | | | |
| | Lactose | — | 50% | 75% |
| | monohydrate | | | |
| Filling | mg | 15.5 | 31 | 31 |
| Dose | mg | 7.75 | 7.75 | 7.75 |
| Analytical results | | | | |
| ED | mg/device | 2.5 | 7.9 | 5.7 |
| FPD | mg/device | 0.3 | 3.9 | 1.8 |
| FPF_{ED} | % | 9.8 | 49.1 | 31.0 |
| MMAD | μm | 4.2 | 2.9 | 3.3 |
| GSD | | 1.8 | 1.9 | 2.0 |
| Device | mg/device | 6.5 | 0.8 | 1.7 |

The results shown confirm that the dosing efficiency is higher for the formulation of the present invention (Composite blend), which also has a higher dissolution rate and higher fine particle dose when compared to the equivalent crystalline form. The formulation of the present invention also has a higher emitted dose and fine particle dose when compared to the spray dried composite particles alone. Furthermore, the formulation of the present invention substantially solves the processability issues seen during filling associated with the high cohesiveness of the spray dried composite active particles.

### Example 2

A formulation was prepared according to the present invention. For preparing the spray dried particles the active ingredient was dissolved and the excipients were dissolved in a suitable solvent. A lab scale spray dryer (Büchi, model B-290), equipped with a two fluid nozzle, was used to atomize and dry the solutions. Co-current nitrogen was used to promote the drying after atomization. The spray drying unit was operated in open cycle mode (i.e., without recirculation of the drying gas). FIG. 1 schematically shows the spray drying set up used. Before feeding the solution to the nozzle, the spray drying unit was stabilized with nitrogen to assure stable inlet (T_in) and outlet temperatures (T_out). After stabilization, the solution was fed to the nozzle by means of a peristaltic pump and atomized at the tip of the nozzle. The droplets were then dried in the spray drying chamber by current nitrogen. The stream containing the dried particles was directed into a cyclone and collected at the bottom. The main operating parameters during the spray drying process are summarized in Table 4.

TABLE 4
Summary of the main operating conditions
Spray solutions

| | | |
|---|---|---|
| Rifampicin (RIF) | 7.5 | g |
| Acetone | 616.5 | g |
| Acetic acid | 16.8 | |
| Trehalose (TRE) | 3.75 | g |
| Water | 151.6 | g |

Spray Drying parameters

| | | |
|---|---|---|
| T_out | 50 | ° C. |
| F_drying (N2) | 40 | kg/h |
| Rotameter level | 50 | mm |

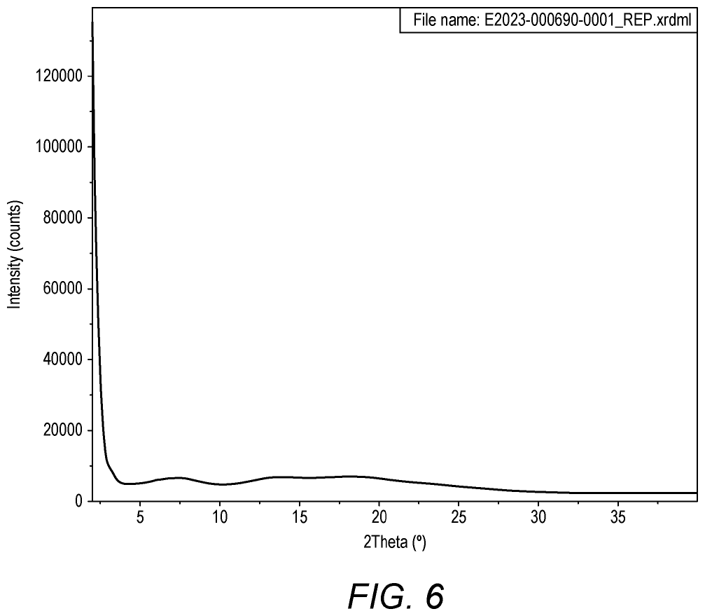

The compound obtained using the method of this invention is an amorphous solid with a higher dissolution rate compared with the corresponding crystalline form. Its amorphous form can be confirmed by x-ray powder diffraction (XRPD). The X-ray powder diffraction pattern of the spray dried composite particles obtained according to the process herein described is presented in FIG. 6.

The composite active particles obtained were blended with coarse lactose, and the formulation composition is shown in Table 5. The spray dried composite particles and the coarse lactose were blended in a low shear blender, in two steps. The first step involved the blending of 66% w/w of total lactose with 50% w/w of the total amount of composite active particles, at 96 rpm, for 5 minutes. The second step involved the addition of the remaining lactose and composite active particles and blending at 96 rpm for 5 minutes more.

TABLE 5
Formulation composition

| Ingredient | Amount (g) | Amount (%) |
|---|---|---|
| Spray dried composite active particles | 4.25 | 85.0 |
| RIF:TRE (67:33 ratio by weight) | | |
| Lactose monohydrate (coarse) | 0.75 | 15.0 |
| TOTAL | 5.00 | 100.0 |

The blend obtained was filled into a reservoir type device. The fill weight used was 20 mg for the composite particle and 30 mg for the carrier-based formulation, in order to maintain the nominal dose. The filling was performed in an auger-filling principle equipment, and no clogging was observed. The aerodynamic performance of spray dried composite particles alone (A) and composite blend (B) (i.e. the spray dried composite particles plus lactose) was assessed through Next Generation Impactor (NGI). The results are presented in Table 6. Although the Emitted dose (ED) is similar, the fine particle fraction (FPF) is higher for the composite blend (B). The fine particle dose (FPD) of the composite active particles alone was 1.5 mg in contrast to the composite blend that was 2.6 mg. The fine particle dose (FPD) of the composite blend is higher than the composite active particles alone. This shows that the dose that reaches the lungs, and will have a therapeutic effect, is higher.

TABLE 6
Aerodynamic performance by NGI

| | |
|---|---|
| A | B |
| Composite | Composite |
| particles | Blend |

| Active material | Composite REM |
|---|---|
| | REM:TRE (67:33) |

Active material particle size
distribution(PSD)
Dv90

| | |
|---|---|
| Dv50 | 2.21.1 |
| Dv10 | 0.5 |

| Composition | | | | |
|---|---|---|---|---|
| | Composition | Composite | 100% | 85% |
| | | particles | | |
| | | Lactose | — | 15% |
| | | monohydrate | | |
| | Filling | mg | 20 | 30 |
| | Dose* | mg | 15.4 | 16.3 |
| Analytical results | | | | |
| | ED | mg/device | 11.7 | 11.9 |
| | FPD | mg/device | 1.5 | 2.6 |
| | FPF_{ED} | % | 12.7 | 21.9 |
| | MMAD | μm | 2.0 | 1.7 |
| | GSD | | 2.0 | 1.7 |
| | Device | mg/device | 3.0 | 4.9 |

*Dose based on powder assay

| | |
|---|---|
| Sample Pan Number | 23 |
| Sample Mass | 5.149 mg |
| Pan Mass | 0.000 mg |
| Reference Pan Number | 54 |
| Reference Sample Mass | 0.000 mg |
| Reference Pan Mass | 53.910 mg |
| Pan Type | Tzero Aluminum |
| Test | Custom |
| Test Name | |
| Segment 1 | Equilibrate 0.00° C. |
| Segment 2 | Modulate Temperature 1.00° C. for 60.0 s |
| Segment 3 | Ramp 2° C./min to 155.00° C. |
| Use Standby Temperature | Yes |
| Discard pan in waste bin | No |
| at end of test | |
| End of Test Use Standby | Yes |
| Temperature | |
| Use default calibrations | Yes |

1-31. (canceled)

32. An inhalable pharmaceutical composition comprising:
(i) spray dried cohesive composite active particles, each composite active particle comprising an active pharmaceutical ingredient (API) material in an amorphous form and an excipient; and
(ii) carrier particles,
wherein the composite active particles and the carrier particles are blended in a physical mixture and wherein the composite active particles comprise 10% to 85% of the pharmaceutical composition by weight.

33. A pharmaceutical composition according to claim 32 wherein the composite active particles have a mass median aerodynamic diameter (MMAD) of equal to or less than 10 μm, or
a mass median aerodynamic diameter (MMAD) of equal to or less than 5 μm.

34. A pharmaceutical composition according to claim 32 comprising one or more of the following features:
(i) wherein the solubility and dissolution rate of the active pharmaceutical ingredient (API) material is higher than that of the crystalline isolated form of the API;
(ii) wherein the composite active particles are more soluble than particles of active pharmaceutical ingredient (API) material alone;
(iii) wherein the composite active particles comprise 10 to 90% of active pharmaceutical ingredient (API) material by weight of the composite active particles.

35. A pharmaceutical composition according to claim 32 wherein the excipient comprises an amino acid, or a sugar, or a mixture of an amino acid and a sugar.

36. A pharmaceutical composition according to claim 35 wherein the amino acid comprises leucine, tryptophan, alanine, valine, isoleucine, trileucine, dileucine, methionine, phenylalanine, or proline or a mixture of two or more thereof.

37. A pharmaceutical composition according to claim 35 wherein the amino acid comprises leucine, isoleucine, trileucine or dileucine or a mixture of two or more thereof.

38. A pharmaceutical composition according to claim 35 wherein the sugar comprises a disaccharide.

39. A pharmaceutical composition according to claim 35 wherein the sugar comprises trehalose, lactose, mannitol, or sucrose or a mixture of two or more thereof.

40. A pharmaceutical composition according to claim 32 wherein the composite active particles comprise about 50 to 85% of the pharmaceutical composition by weight.

41. A pharmaceutical composition according to claim 32 wherein the carrier particles are selected from lactose, mannitol, trehalose, raffinose, sucrose, microcrystalline cellulose or a mixture of two or more thereof; or wherein,
the carrier particles comprise lactose or lactose monohydrate, or a mixture of lactose and lactose monohydrate.

42. A pharmaceutical composition according to claim 32 wherein the carrier particles comprise about 15% to 90% of the pharmaceutical composition by weight, or
wherein the carrier particles comprise about 15% to 50% of the pharmaceutical composition by weight.

43. A pharmaceutical composition according to claim 32 wherein the carrier particles have a mass median aerodynamic diameter (MMAD) of greater than 25 μm; or
wherein the carrier particles have a mass median aerodynamic diameter (MMAD) of from 50 μm to 100 μm.

44. A process of preparing a pharmaceutical composition according to claim 32 comprising the steps of:
i) providing a solution of an active pharmaceutical ingredient (API) material and a solution of an excipient, which may be separate solutions or a combined solution, and spray drying to provide cohesive composite active particles, wherein each composite active particle comprises an active pharmaceutical ingredient (API) material in an amorphous form and an excipient;
ii) blending the spray dried cohesive composite active particles with carrier particles to form a physical mixture and wherein the composite active particles comprise 10% to 85% of the pharmaceutical composition by weight.

45. A process of preparing a pharmaceutical composition according to claim 44, wherein after step (i) or after step (ii), or both, a post drying or conditioning step is performed.

46. A process of preparing a pharmaceutical composition according to claim 44, wherein the composite active particle size distribution is controlled to a Dv50 of equal to or <5 μm.

47. A process of preparing a pharmaceutical composition according to claim 44 wherein in step (ii) the blending comprises a high shear or a low shear process.

48. A blister or series of blisters, or one or more capsules, for use in a dry powder inhaler, comprising a pharmaceutical composition according to claim 32.

49. A dry powder inhaler comprising a blister or series of blisters, or one or more capsules, according to claim 48.

50. A dry powder inhaler which is a reservoir device, comprising a pharmaceutical composition according to claim 32.

51. A method of treating a pulmonary condition, said method comprising administering to a patient in need thereof a pharmaceutical composition according to claim 32.
